(19) United States
(12) Patent Application Publication
(10) Pub. No.: US 2009/0292219 A1
(43) Pub. Date: Nov. 26, 2009

(54) REAL TIME DETECTION OF UPPER RESPIRATORY SYSTEM CONDITIONS IN HORSES

(76) Inventors: John Pringle, Knivsta (SE); Miia Riihimäki, Uppsala (SE)

Correspondence Address: PORTER WRIGHT MORRIS & ARTHUR, LLP;INTELLECTUAL PROPERTY GROUP, 41 SOUTH HIGH STREET, 28TH FLOOR, COLUMBUS, OH, 43215, US

(21) Appl. No.: 12/306,551

(22) PCT Filed: Jun. 28, 2007
(86) PCT No.: PCT/IB07/01966

§ 371 Date: Dec. 24, 2008

Related U.S. Application Data
(60) Provisional application No. 60/818,108, filed on Jun. 30, 2006.
(60) Provisional application No. 60/837,463, filed on Aug. 11, 2006.
(60) Provisional application No. 60/839,566, filed on Aug. 23, 2006.

(51) Int. Cl. A61B 5/08

(52) U.S. Cl. 600/529

(57) ABSTRACT

Devices and methods allow for real time detection of an upper respiratory system condition in a horse. The device comprises a monitor of a size permitting positioning of the monitor in an upper throat region of a horse via a nasal cavity, and a sleeve operable to insert the monitor into and position the monitor within an upper throat region of a horse via a nasal cavity. The device may further include a transmitter operable to wirelessly transmit video signals and optionally sound signals from the monitor to a receiver remote from the horse. The method comprises positioning the monitor in an upper throat region of a horse via a nasal cavity, subjecting the horse to a workout, and obtaining with the monitor visual events and optionally sound events in the upper throat region of the horse during the workout. The method may further comprise transmitting video signals and optionally sound signals from the monitor to a receiver remote from the horse during the workout.

### FIELD OF THE INVENTION

The present invention is directed to devices and methods which allow real time detection of the upper respiratory system conditions in a horse. Because the devices and methods of the invention allow detection of such conditions under normal workout procedures, more accurate and informative information regarding the upper respiratory system, and, for example, disease and/or abnormalities thereof, can be obtained.

### BACKGROUND OF THE INVENTION

Horse sports are a popular free time activity in Europe, North America and elsewhere. Most horse competitions or leisure activities require the horses to perform as athletes, and horse owners are typically keenly observant when their horses do not perform to expectations. They often look to their veterinarian for diagnosis and possible correction of problems that impair the athletic ability of their horses, and horse owners worldwide have an ever increasing demand for a high level of medical care and competence in the diagnosis and treatment of ailments in their horses. One of the most common reasons horse owners seek veterinary assistance is when their animal fails to perform to expectations with no obvious cause or when a horse begins to make an abnormal sound while breathing during exercise.

In both instances, a major source of the problem that must be investigated is the upper airway throat structures, including the pharynx, larynx, and soft palate. If the upper airway throat structures are abnormal or function inappropriately during exercise, a bottleneck to airflow from the nose to the lungs can result. Significantly, many upper airway problems occur only at the very highest intensity of work, when the airflow is greatest and the horse is reaching its threshold of fatigue. There are a number of various upper airway abnormalities that can cause the same general signs of failure to perform athletically, and for each there are specific medical treatments, either surgical or pharmaceutical, although in some cases there are no known effective treatments. Thus, accurate diagnosis is vital for providing medical advice to owners regarding optimal treatment and eventual prognosis.

A current method to examine a horse for upper airway problems demands that the horse run at high speeds on a specially designed equine treadmill while the horse's throat is examined visually using a video endoscope that is placed via the nasal cavity into the upper airway cavity. This type of examination requires that the horse first be trained to run on the treadmill, and that it then perform a physical workout on the treadmill with an endoscope placed in the upper airway. At least one assistant is needed to stand near the head of the horse to hold the body of the endoscope away from the horse while the horse works at high speed. This procedure is currently accepted as the “Gold Standard” for evaluating horses with suspected upper airway problems that impair performance.

However, the described method has several substantial drawbacks. First, it requires the use of comparatively cumbersome and expensive equipment, namely, a high speed equine treadmill along with a video endoscope. Additionally, the procedure is time and labor intensive, requiring training of the horse to adjust to the unfamiliar working conditions of running on a treadmill, and at the same time, having an endoscope placed in the throat. In fact, more high spirited horse breeds refuse to accept such conditions and show signs of panic or fright, risking injury to the horse and/or assistants. Moreover, the procedure requires the participation of at least two or more trained assistants to help the veterinarian in safely conducting the examination. However, by far the largest drawback is that the horse does not workout under natural “real life” conditions, with the head carriage and position and the type and effort of work performed failing to mirror real life working conditions in which the upper airway problem occurs. Because of these factors, the examination on the treadmill frequently fails to recreate the conditions related to a horse's poor field performance, and the veterinarian, despite all efforts, fails to establish a proper diagnosis of an actual upper airway problem.

Recent research on methods to record sound produced by a horse when it works at maximal effort and research on sound frequencies produced when various upper airway abnormalities occur, have shown some promise for diagnosis. For example, see Derksen et al, AJVR (2001) and Franklin et al, EVJ (2003). However, the former study did not correlate sound production to any visual confirmation of an abnormality or upper airway disease during the sound recording, and the latter study employed examination on treadmills and thus retained the disadvantages described above associated with the use of an equine treadmill.

Accordingly, there is a significant need for improved methods and devices for diagnosis of upper airway/respiratory system conditions in horses.

### SUMMARY OF THE INVENTION

The present invention is directed to methods and devices for improved detection of an upper respiratory system condition in a horse.

In one embodiment, the invention is directed to a device for real time detection of an upper respiratory system condition in a horse. The device comprises a monitor of a size permitting positioning of the monitor in an upper throat region of a horse via a nasal cavity, wherein the monitor is operable to obtain visual events in an upper throat region of a horse, and a sleeve operable to insert the monitor into and position the monitor within an upper throat region of a horse via a nasal cavity. In further embodiments, the device includes a transmitter operable to wirelessly transmit video and signals from the monitor to a receiver remote from the horse, a storage device operable to store video signals from the monitor, and/or a video display directly linked to the monitor and operable to receive and display video signals from the monitor in real time.

In another embodiment, the invention is directed to a method for real time detection of an upper respiratory system condition in a horse. The method comprises positioning a monitor in an upper throat region of a horse via a nasal cavity, wherein the monitor is operable to obtain visual events in the upper throat region of the horse, subjecting the horse to a workout, and obtaining with the monitor visual events in the upper throat region of the horse during the workout. In further embodiments, the method further comprises transmitting video signals from the monitor to a receiver remote from the horse during the workout, storing video signals from the monitor on a storage device, and/or displaying video signals from the monitor on a directly linked video display.

In a further embodiment, the invention is directed to a device for real time detection of an upper respiratory system condition in a horse. The device comprises a monitor of a size permitting positioning of the monitor in an upper throat region of a horse via a nasal cavity, wherein the monitor is operable to obtain visual and sound events in an upper throat region of a horse, a sleeve operable to insert the monitor into and position the monitor within an upper throat region of a horse via a nasal cavity, and a transmitter operable to wirelessly transmit video and sound signals from the monitor to a receiver remote from the horse.

In yet another embodiment, the invention is directed to a method for real time detection of an upper respiratory system condition in a horse. The method comprises positioning a monitor in an upper throat region of a horse via a nasal cavity, wherein the monitor is operable to obtain visual and sound events in the upper throat region of the horse, subjecting the horse to a workout, obtaining with the monitor visual and sound events in the upper throat region of the horse during the workout, and transmitting video and sound signals from the monitor to a receiver remote from the horse during the workout.

The devices and methods of the invention are advantageous in that they provide diagnosis of the condition of a horse's upper respiratory system under real physical workout conditions, and do not require the cumbersome equine treadmill or endoscope employed in the aforementioned conventional procedures. Additional advantages of the devices and methods of the invention will be further apparent in view of the following detailed description.

### BRIEF DESCRIPTION OF THE DRAWINGS

The following detailed description will be more fully understood in view of the drawing in which:

FIG. 1 shows a schematic view of a first embodiment of a device according to the invention;

FIG. 2 shows an enlarged schematic view of a first embodiment of a suitable monitor included in a device according to the invention;

FIG. 3 shows the device of FIG. 1 laid upon a model of an upper respiratory system of a horse;

FIG. 4 shows a schematic view of a device according to the invention in use;

FIG. 5 shows a schematic view of a second embodiment of a device according to the invention;

FIG. 6 shows a schematic view of a third embodiment of a device according to the invention;

FIG. 7 shows an enlarged schematic view of a second embodiment of a suitable monitor included in a device according to the invention;

FIG. 8 shows the device of FIG. 6 laid upon a model of an upper respiratory system of a horse; and

FIG. 9 shows a halter attachment for attaching and supporting one or more external components of a device according to the invention adjacent the head of a horse.

The embodiments set forth in the drawing are illustrative in nature and are not intended to be limiting of the invention defined by the claims. Moreover, individual features of the drawing and the invention will be more fully apparent and understood in view of the detailed description.

### DETAILED DESCRIPTION

The present invention is directed to devices and methods which allow real time detection of the upper respiratory system conditions in a horse. Because the devices and methods of the invention allow detection of such conditions under normal workout procedures, more accurate and informative information regarding the upper respiratory system, and, for example, abnormalities thereof, can be obtained.

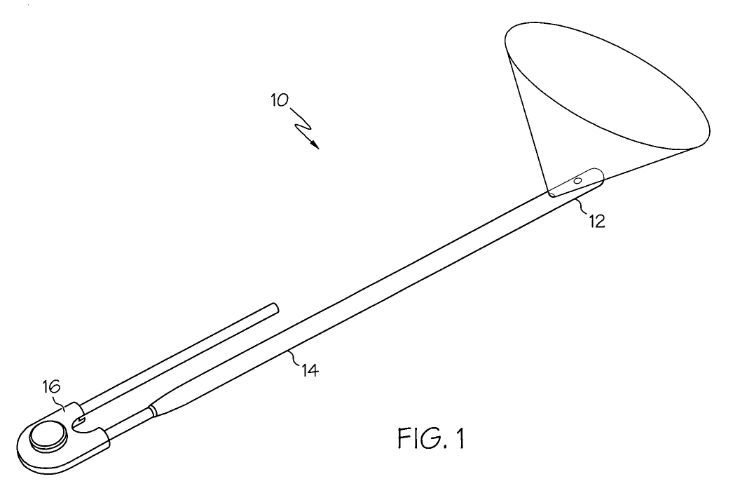

With reference to FIG. 1, in one embodiment, a device 10 according to the invention comprises a monitor 12, a sleeve 14 and a transmitter 16. The monitor 12 is of a size permitting positioning of the monitor in an upper throat region of a horse via a nasal cavity, and the monitor is operable to obtain visual, and optionally sound, events in an upper throat region of a horse. The monitor enables both picture and optionally sound capture of the events in the upper airway in the horse under intense work effort. Instead of being linked to treadmill-based observations, as the conventional method described above, the present device captures similar information in the natural working setting, unrestricted by a stationary treadmill and video endoscope equipment. As will be appreciated, and described in further detail below, the monitor can be placed in close proximity to the area for which examination and monitoring is desired and can be fixed in place, either by the constraints of the location or with additional locating devices.

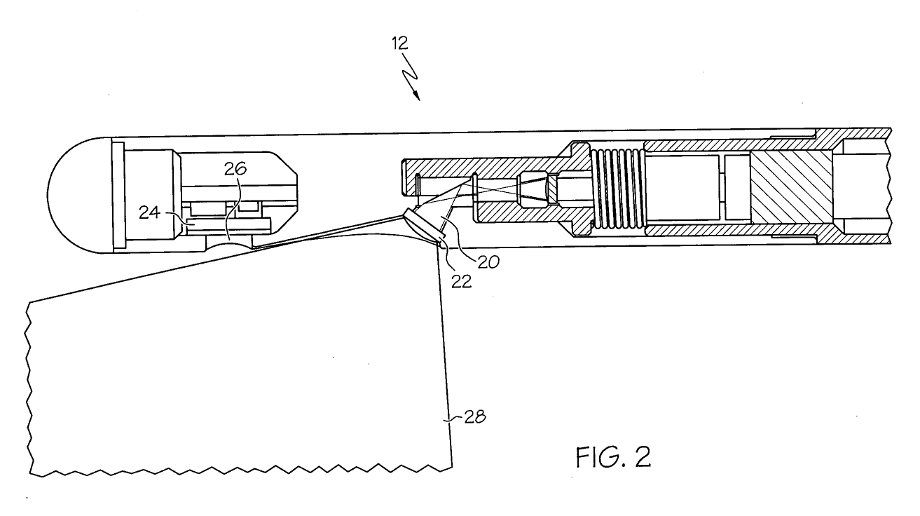

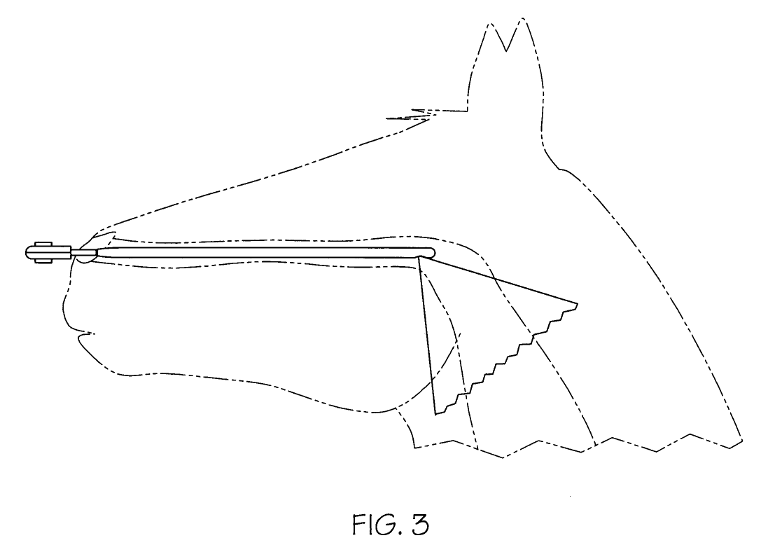

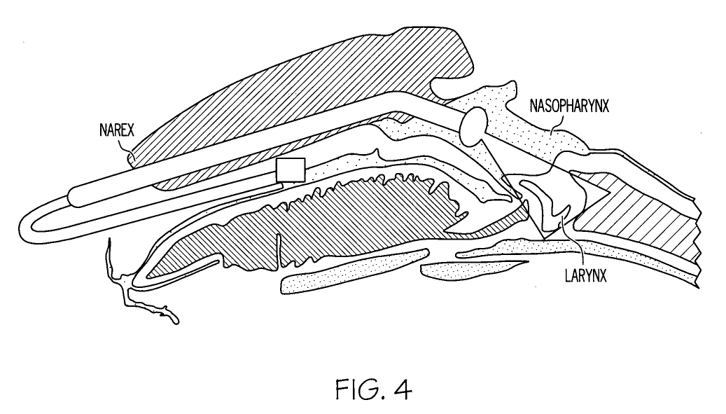

As shown in FIG. 2, in one embodiment, the monitor 12 comprises a camera 20, including a lens 22, with video capabilities, for example CMOS (complementary metal oxide semiconductor), CCD (charge coupled device), or other video/imaging capacity, and a light source 26 for illuminating an area to be examined, and, optionally, related physiologic sensors. If it is desirable to capture sound events as well as video events, the monitor 12 may further include a microphone 24. The sleeve 14 is operable to insert the monitor into and position the monitor within an upper throat region (pharynx) of a horse via a nasal cavity. A trained veterinarian may easily properly locate the monitor via the nasal cavity in the upper throat region using the sleeve. The monitor is sized to fit within the inner limits of the nasal cavity and, in one embodiment, is positioned at the end of the sleeve with the camera, light source and microphone, if included, positioned at the inner tip to capture visual and sound events at the inner throat cavity (nasopharynx) as shown in FIGS. 3 and 4. More specifically, the camera, light source and microphone may be positioned overlooking the pharyngeal cavity as shown in FIGS. 3 and 4.

In a specific embodiment, the sleeve has a diameter less than about 20 mm and a length of less than about 700 mm. In a more specific embodiment, the sleeve has a diameter of from about 10-20 mm and a length of from about 300-700 mm to fit snugly within the nasal cavity of the horse but not protrude externally so that it can remain in the nasal cavity without further manipulation or adjustment while the horse performs its natural workout. Suitably, the camera will include a lens having a field of view 28 as shown in FIG. 2 sufficient to view areas of interest in the upper respiratory system of a horse. In a specific embodiment, the camera includes a wide angled lens. In a more specific embodiment, the lens has a focus of from about 10 mm to about 200 mm, and in a yet more specific embodiment, the lens has a focus of from about 10 to about 150 mm.

The light source preferably is a bright but cold light source so that the area of interest, and particularly the field of view of the camera lens, is adequately illuminated yet not subject to potentially tissue-damaging heat. In a specific embodiment, the light source is a light emitting diode (LED). One of ordinary skill in the art may design a field of illumination as desired.

All parts of the device are preferably washable. In more specific embodiments, the monitor and sleeve may advantageously be provided with a body surface which is smooth, scratch resistant and washable, and preferably sterilizable, to avoid mechanical trauma to the inner nasal cavity membranes and to prevent transmission of infectious agents between horses being examined. Further, the sleeve may be formed a of a material having a desirable balance of rigidity and flexibility to allow insertion in the nasal cavity as desired. Various plastic and metal materials are suitable in this regard. Examples include, but are not limited to titanium, aluminum, stainless steel, biocompatible polymers such as polyethylene, polysilioxanes, polyacrylates and polymethacrylates, and the like, and composite polymers.

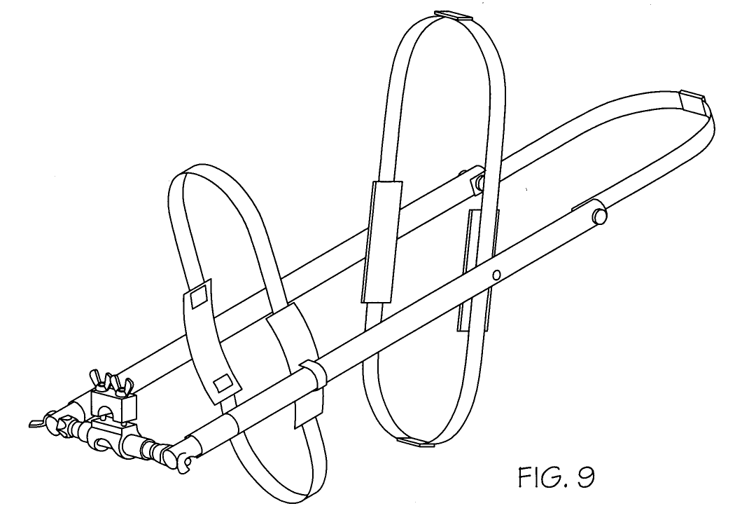

The transmitter 16 is operable to wirelessly transmit video recording signals, and optionally sound recording signals if desired, from the monitor to a receiver which is located remote from the horse. In the embodiment shown in FIG. 1, the transmitter 16 is connected with the monitor 12 via one or more electrical cables within the sleeve 14. Examples include, but are not limited to, cables having a diameter of about 2-5 mm. Suitably, the electric cables include both audio and video feed capabilities. The transmitter is, in this embodiment, adapted for location external to a horse. For example, the transmitter may be adapted for location on a halter or other accessory, as desired. An exemplary halter is shown in FIG. 9. It will be evident that once set in place, the device according to the invention will not be of undue distraction to a horse during exercise so that the horse may work at maximal intensity in field conditions with full freedom of movement.

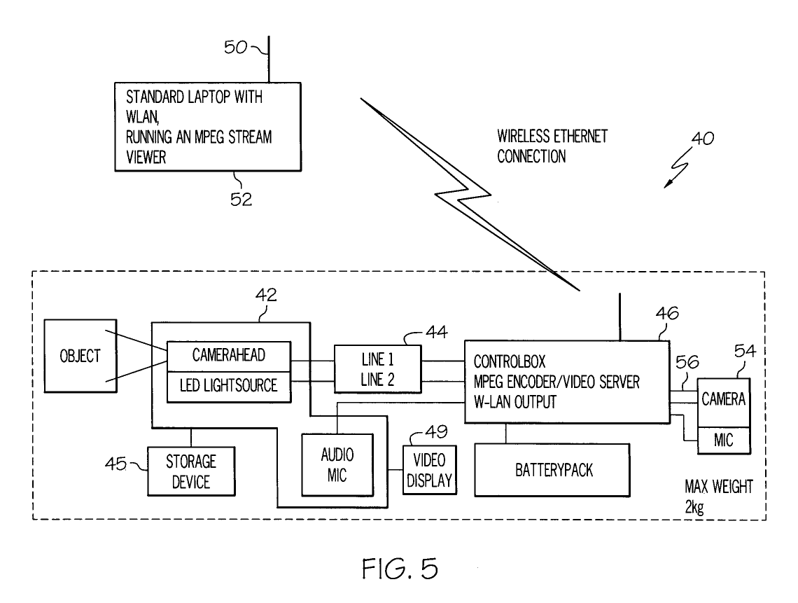

FIG. 5 shows a schematic view of one embodiment of a device according to the invention. The device 40 includes a monitor 42, a sleeve 44 and a transmitter 46, with the monitor 42 including a camera, LED light source and microphone, connected via cables within the sleeve 44 to the transmitter 46. As shown, the transmitter may include controls, together with an MPEG encoder/video server and wireless local area networking (W-LAN) output function. Optionally, the transmitter may include a battery pack for independent operation. In this embodiment, the device further includes a receiver 50 operable to receive video and audio signals from the transmitter. In one embodiment, the transmitter and receiver form a wireless ethernet connection. Advantageously, the receiver 50 may be in communication with a computer 52 for video and audio broadcasting signals received by the receiver. Thus, images and sound can be viewed and heard in real time.

In further embodiments, the device of the invention may include a storage device 47, in addition to or in place of, the transmitter 46. The storage device is operable to electronically store the video signals, and optionally audio signals, from the monitor. The storage device may be accessed or removed in order to access and/or download the signals stored thereon upon removal of the device from the subject horse. One of ordinary skill will appreciate that various electronic storage devices as described are commercially available and suitable for use herein. The device may also include, in addition to or in place of the transmitter, a video display 49 directly linked to the monitor and operable to receive and display video, and optionally audio, signals from the monitor in real time. Such a display may assist a rider during a workout to obtain desired diagnostic conditions and/or preserve the well being of the subject horse.

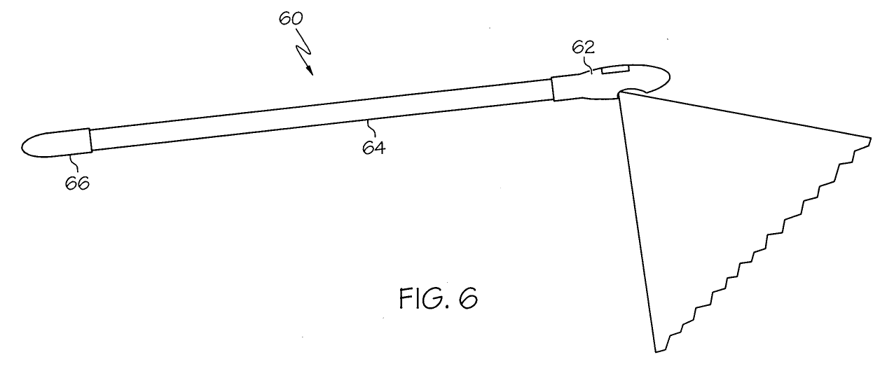

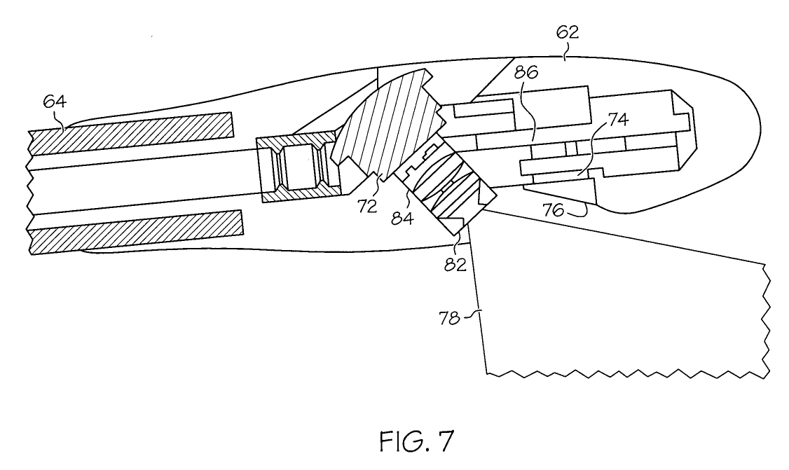

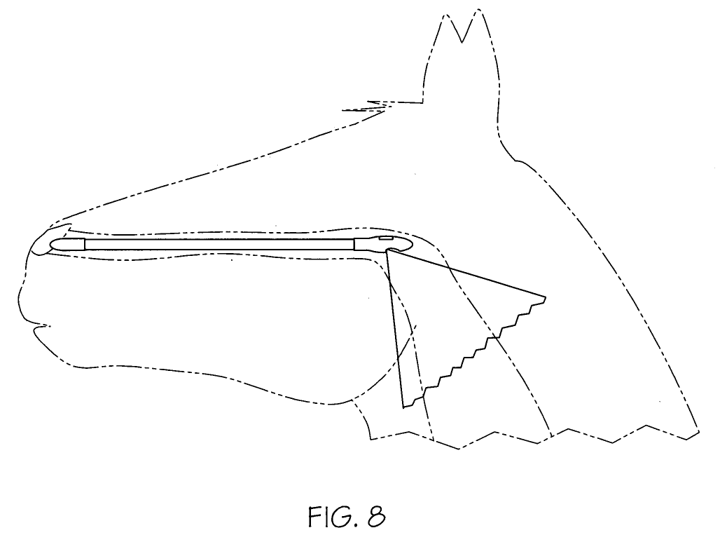

FIGS. 6-8 show a further embodiment of a device 60 according to the invention in the form of a single elongated member comprising a monitor 62 at one end, a sleeve 64, and a transmitter 66 at the opposite end thereof. As shown in FIG. 7, the monitor 62 includes a camera 72, having a field of view 78, a microphone 74 and a light source 76. The camera may, for example, include a lens component 82 and a charge coupled device (CCD) camera unit 84 which uses a small, rectangular piece of silicon rather than a piece of film to receive incoming light. The microphone 74 and the light source 76, for example an LED, are conveniently provided on a single printed circuit board (PCB) 86. FIG. 8 shows placement of the device laid upon a model of an upper respiratory system of a horse.

Thus, according to the invention, a method for real time detection of an upper respiratory system condition in a horse is provided. A monitor is positioned in an upper throat region of a horse via a nasal cavity and is operable to obtain visual and optionally sound events in the upper throat region of the horse. The horse is subjected to a workout, and visual events, and optionally sound events, are obtained in the upper throat region of the horse during the workout and transmitted from the monitor to a receiver remote from the horse during the workout. The images and dynamic physiologic events which are captured from the upper airway of the horse during intense exercise are transferred by commercially available wireless technology to a remote site with the capacity for both real time and after workout analysis of the dynamic upper airway events. Study of the real time video images and sound of a horse's upper respiratory function during a workout allows coupling of a suspected upper airway problem to direct recording of airway structure and sound at the very time they occur, and under the most natural workout conditions possible. The invention can therefore greatly improve the ease and accuracy of diagnostic upper airway studies on horses when compared with currently-available state of the art equipment.

Advantageously, introduction of the device according to the invention to obtain real time video and sound created during dynamic function of the upper airway system of the horse overcomes the labor and resource intensive requirements encountered when conducting diagnostic tests with a stationary video endoscope and an equine high speed treadmill. The devices and methods of the invention thus save time and resources as fewer persons are needed to conduct a diagnostic test and less time is needed to train the horse to accept the instrumentation. Moreover, risk to injury of a horse or personnel is reduced by avoiding the need for intense exercise by the horse in a laboratory setting. Rather, the horse is allowed to conduct its customary workout effort under familiar and natural conditions. Most importantly, capturing the dynamic events remotely through a miniature “hidden” monitor allows capture of dynamic changes in the upper airway system of the horse, uncontaminated by forcing the horse to work in an artificial lab setting, and can thereby provide a more reliable representation of upper airway health, disease and/or abnormalities in the horse. As well, by combining sound capture with the video capture of the airway function, the monitor provides a direct relationship of various anatomical abnormalities to sound production, and vice versa, where an abnormal respiratory sound is the item of concern.

In a further embodiment, the device as described herein may further include one or more monitors adapted for placement at a position external to the horse, for example on a halter or other accessory, to provide additional monitoring of conditions external to the horse. The external monitor is operable to obtain visual and optionally sound events external to the horse and the aforementioned transmitter is operable to wirelessly transmit video and optionally sound signals from the external monitor to the receiver remote from the horse. For example, with reference to FIG. 5, the device 40 further includes an external monitor 54 connected via cables 56 to the transmitter 46. The external monitor 54 suitably includes a camera, optionally a light source, such as an LED light source which illuminates the desired external area without generating tissue-damaging heat, and optionally a microphone. The receiver 50 which receives video and audio broadcasting signals and the computer 52 in communication with the receiver 50 will distinguish between video and audio signals from the internally placed monitor 42 and the external monitor 54. Additionally, the receiver and/or the computer can assemble both video and sound images as desired for providing an overall evaluation of the monitored conditions, both internally and externally as desired.

In a further embodiment, the external monitor is positioned at a location sufficiently remote from the horse in order to provide a view of the entire horse during the workout whereby all of the horse's motions may be viewed in conjunction with the real time monitoring of the upper respiratory system.

The device according to the present invention may further be provided with additional monitoring capabilities. For example, in one embodiment, the device may include, or be used in combination with, a pulsoximeter, which is an instrument for monitoring the blood oxygenation, and/or a heart rate monitor. Additional physiological data from the monitored subject may also be collected, as desired.

The device of the invention may further include additional operability to facilitate monitoring, treatment, and/or the well being of the horse. In one embodiment, the device may include a global positioning system (GPS) for facilitating data collection and review at a location remote from the testing site. In another embodiment, the device may include a video and/or sound display operable to communicate in real time with a horse rider, driver or jockey. Further, in embodiments of the device including a computer, for example computer 52 as shown in FIG. 5, the computer may be provided with one or more software programs for diagnostic assessment of the video and optionally sound signals. In conjunction with a diagnostic assessment program, or as an alternative to a diagnostic assessment program, the computer may be provided with a database of reference images, videos and/or sound recordings to assist in diagnosis of various clinical conditions. Further, the computer may include storage means for storage of collected data, particularly associated with respective patients. In this regard, the software system may include evaluation and/or certification operability for insurance purposes.

The specific illustrations and embodiments described herein are exemplary only in nature and are not intended to be limiting of the invention defined by the claims. Further embodiments and examples will be apparent to one of ordinary skill in the art in view of this specification and are within the scope of the claimed invention.

What is claimed is:

1. A device for real time detection of an upper respiratory system condition in a horse, comprising a monitor of a size permitting positioning of the monitor in an upper throat region of a horse via a nasal cavity, wherein the monitor is operable to obtain visual and sound events in an upper throat region of a horse, a sleeve operable to insert the monitor into and position the monitor within an upper throat region of a horse via a nasal cavity, and a transmitter operable to wirelessly transmit video and sound signals from the monitor to a receiver remote from the horse.

2. The device of claim 1, wherein the monitor includes a camera, a light source and a microphone.

3. The device of claim 2, wherein the light source is operable to illuminate an upper throat region of a horse to enable capture of visual events by the monitor therein without generating tissue-damaging heat.

4. The device of claim 2, wherein the light source comprises a light emitting diode.

5. The device of claim 1, wherein the sleeve has a diameter less than about 20 mm and a length of less than about 700 mm.

6. The device of claim 1, wherein the sleeve has a diameter of from about 10-20 mm and a length of from about 300-700 mm.

7. The device of claim 1, wherein the monitor and the sleeve have outer surfaces which are sterilizable.

8. The device of claim 1, wherein the transmitter is adapted for location external to a horse.

9. The device of claim 8, wherein the transmitter is adapted for location on a halter.

10. The device of claim 1, further comprising a receiver operable to receive video and audio signals from the transmitter.

11. The device of claim 10, further comprising a computer for video and audio broadcasting signals received by the receiver.

12. A method for real time detection of an upper respiratory system condition in a horse, comprising positioning a monitor in an upper throat region of a horse via a nasal cavity, wherein the monitor is operable to obtain visual and sound events in the upper throat region of the horse, subjecting the horse to a workout, obtaining with the monitor visual and sound events in the upper throat region of the horse during the workout, and transmitting video and sound signals from the monitor to a receiver remote from the horse during the workout.

13. The method of claim 12, wherein the signals received by the receiver are video and audio broadcast by a computer in communication with the receiver.

14. The method of claim 12, wherein the monitor is positioned within the upper throat region of the horse by a sleeve to which the monitor is attached.

15. The method of claim 14, wherein the sleeve has a diameter less than about 20 mm and a length of less than about 700 mm.

16. The method of claim 14, wherein the sleeve has a diameter of from about 10-20 mm and a length of from about 300-700 mm.

17. The method of claim 12, wherein the monitor includes a camera, a light source and a microphone.

18. A device for real time detection of an upper respiratory system condition in a horse, comprising a monitor of a size permitting positioning of the monitor in an upper throat region of a horse via a nasal cavity, wherein the monitor is operable to obtain visual events in an upper throat region of a horse, and a sleeve operable to insert the monitor into and position the monitor within an upper throat region of a horse via a nasal cavity.

19. The device of claim 18, further comprising a transmitter operable to wirelessly transmit video signals from the monitor to a receiver remote from the horse.

20. The device of claim 19, wherein the monitor includes a camera and a light source operable to illuminate an upper throat region of a horse to enable capture of visual events by the monitor therein without generating tissue-damaging heat.

21. The device of claim 18, further comprising a storage device operable to record video signals from the monitor.

22. The device of claim 21, wherein the monitor includes a camera and a light source operable to illuminate an upper throat region of a horse to enable capture of visual events by the monitor therein without generating tissue-damaging heat.

23. The device of claim 18, wherein the monitor includes a camera and a light source operable to illuminate an upper throat region of a horse to enable capture of visual events by the monitor therein without generating tissue-damaging heat.

24. The device of claim 23, wherein the sleeve has a diameter of from about 10-20 mm and a length of from about 300-700 mm.

25. The device of claim 18, further comprising a video display directly linked to the monitor and operable to receive and display video signals from the monitor in real time.

26. A method for real time detection of an upper respiratory system condition in a horse, comprising positioning a monitor in an upper throat region of a horse via a nasal cavity, wherein the monitor is operable to obtain visual events in the upper throat region of the horse, subjecting the horse to a workout, and obtaining with the monitor visual events in the upper throat region of the horse during the workout.

27. The method of claim 26, further comprising transmitting video signals from the monitor to a receiver remote from the horse during the workout.

28. The method of claim 26, further comprising storing video signals from the monitor on a storage device.

29. The method of claim 26, further comprising displaying video signals from the monitor on a directly linked video display.
